(19) United States
(12) Patent Application Publication
(10) Pub. No.: US 2007/0280967 A1
(43) Pub. Date: Dec. 6, 2007

(54) Cryo-protective agents for microorganisms

(76) Inventor: Tim Lee, Toronto (CA)

Correspondence Address: Reza, Yacoob, Sanfoi, Pasteur Limited, 1755 Steeles Avenue West, Toronto, ON, M2R 3T4, CA

(21) Appl. No.: 10/581,378

(22) PCT Filed: Nov. 30, 2004
(86) PCT No.: PCT/CA04/02025

§ 371 Date: May 17, 2007

Related U.S. Application Data
(60) Provisional application No. 60/481,734, filed on Dec. 3, 2003.

(51) Int. Cl. A61K 39/05; C12N 1/20

(52) U.S. Cl. 424/245.1; 435/252.1

(57) ABSTRACT

A lyophilization medium fora microorganism is provided wherein the medium is substantiality free of animal-derived products and comprises yeast extract and monosodium glutamate. The lyophilisation medium can be used for cryoprotection of strains of bacteria such as Corynebacterium diphtheriae. Method for preparing a freeze-dried culture of a microorganism using the lyophilization medium, and lyophiles of microorganisms are also provided.

### FIELD OF THE INVENTION

The present invention relates to cryo-protective agents for microorganisms.

### BACKGROUND OF THE INVENTION

Vaccines are often produced by growing a pathogen in a culture medium, isolating the pathogen or a portion of the pathogen or a product of the pathogen and using this material as an immunogen for formulating a vaccine. Vaccines containing whole pathogens include whole cell pertussis vaccines and measles vaccines. Vaccines containing portions of the pathogen include acellular pertussis vaccines. Vaccines containing a product of the pathogen include diphtheria and tetanus vaccines. The pathogen, portion or product may require detoxification by for example chemical treatment before it can be used as a vaccine.

An example of a pathogen from which a product is used in the production of a vaccine is Corynebacterium diphtheriae and the product is diphtheria toxin. Diphtheria is a life-threatening disease caused by infection with C. diphtheriae, a gram-positive, aerobic, rod-shaped bacterium. The disease is caused by local invasion of nasopharyngeal tissues by toxin-producing strains of C. diphtheriae. The organisms grow in a tough, fibrinous membrane overlying a painful, hemorrhapic, and necrotic lesion, which may be located on the tonsils or within the nasopharynx region. During typical epidemics of the past, the spread of the disease was by droplet infection. Patients who recover from diphtheria may carry toxigenic bacteria in their throats and nasopharynx for weeks or months, unless intensively treated with antibiotics.

Most of the clinical symptoms of diphtheria are due to the potent diphtheria toxin produced from corynebacterioprophage carrying the tox gene. After the prophage infects the C. diphtheriae strain and lysogenization has taken place, the strain becomes virulent. Toxin neutralizing antibodies (antitoxin) induced by active immunization with non-toxic forms (toxoids) of the diphtheria toxin can prevent diphtheria. The current immunization strategy is the utilization of diphtheria vaccines prepared by converting the diphtheria toxin into its non-toxic, but antigenic, toxoid form by formaldehyde treatment. The diphtheria toxoid is used in various combinations with other vaccine components for mass immunization worldwide. The World Health Organization (WHO) recently estimated that about 100,000 cases worldwide and up to 8,000 deaths per year are due to decreased immunization of infants, waning immunity to diphtheria in adults and insufficient supply of vaccines.

The variant of the Parke Williams 8 (PW8) strain of Corynebacterium diphtheriae is often used to produce the exotoxin from which the toxoid is prepared by chemical modification. In general, a medium formulation with amino acids, trace vitamins, inorganic salts and a carbohydrate source such as maltose promotes excellent growth of the bacterium. Different media, such as the acid digest of casein and the enzymatic digest of beef muscle (trypsin or papain) are suitable media for toxin production. In conventional methods, the bacteria are cultivated in media containing proteinaceous material of animal origin. A commonly used medium in diphtheria production is the NZ-Amine Type A medium, which contains a casein digest. Under optimal conditions, the amount of toxin produced using NZ-Amine Type A media is 180 Lf/mL using the Limes of flocculation method.

The use of proteinaceous material of animal origin in the production of vaccines such as the exemplified diphtheria vaccine can result in the introduction of undesirable contaminants into the diphtheria toxin produced using such a medium.

Most workers have concentrated efforts on the production of growth media substantially free or devoid of animal-components for the cultivation of pathogens such as C. diphtheriae. There is also a need to provide seed cultures and in particular cryoprotective agents substantially free or devoid of animal-components for microorganisms including pathogens such as C. diphtheriae.

### SUMMARY OF THE INVENTION

The present invention is concerned with cryo-protective agents for microorganisms.

In one aspect of the invention, there is provided a lyophilization medium for a microorganism wherein the medium is substantiality free of animal-derived products and comprises yeast extract and monosodium glutamate. The lyophilization medium may comprise about 1-10% (w/v) monosodium glutamate and about 1-10% (w/v) yeast extract such as about 5% (w/v) monosodium glutamate and about 10% (w/v) yeast extract. The microorganism may be a strain of bacteria including Corynebacterium diphtheriae.

In a second aspect of the invention, there is provided a method for preparing a freeze-dried culture of a microorganism comprising the steps of providing a quantity of the microorganism, mixing said quanity with a lyophilization medium wherein the medium is substantiality free of animal-derived products and comprises yeast extract and monosodium glutamate to provide a mixture and freeze-drying said mixture. The lyophilization medium may comprise about 5% (w/v) monosodium glutamate and about 10% (w/v) yeast extract such as about 5% (w/v) monosodium glutamate and about 10% (w/v) yeast extract. The freeze-drying of said mixture may comprise steps of achieving a first temperature of about −30° C. for said mixture to provide a cooled mixture and maintaining said cooled mixture in a vacuum for a time until said cooled mixture is substantially dry to provide a dried mixture. Suitable vacuums are about 120 mT and suitable times are between about 10 and about 12 hours. The step of maintaining the cooled mixture in a vacuum for a time until said cooled mixture is substantially dry to provide a dried mixture may comprise maintaining said cooled mixture in a vacuum for a time of between about 10 and about 12 hours and increasing said temperature of about −30° C. to a second temperature of about +20° C. Suitable vacuums are about 120 mT. The microorganism may be a strain of bacteria including Corynebacterium diphtheriae.

There is also provided a freeze-dried lyophile comprising cells of a microorganism and a lyophilization medium wherein the medium is substantiality free of animal-derived products and comprises yeast extract and monosodium glutamate. The lyophilization medium may comprise about 1-10% (w/v) monosodium glutamate and about 1-10% (w/v) yeast extract such as about 5% (w/v) monosodium glutamate and about 10% (w/v) yeast extract. The microorganism may be a strain of bacteria including Corynebacterium diphtheriae.

### BRIEF DESCRIPTION OF DRAWINGS

The present invention will be futher understood from the following description with reference to the drawing, in which:

FIG. 1 shows a flow diagram outlining the preparation and lyophilization of a C. diphtheriae culture.

### DETAILED DESCRIPTION OF THE INVENTION

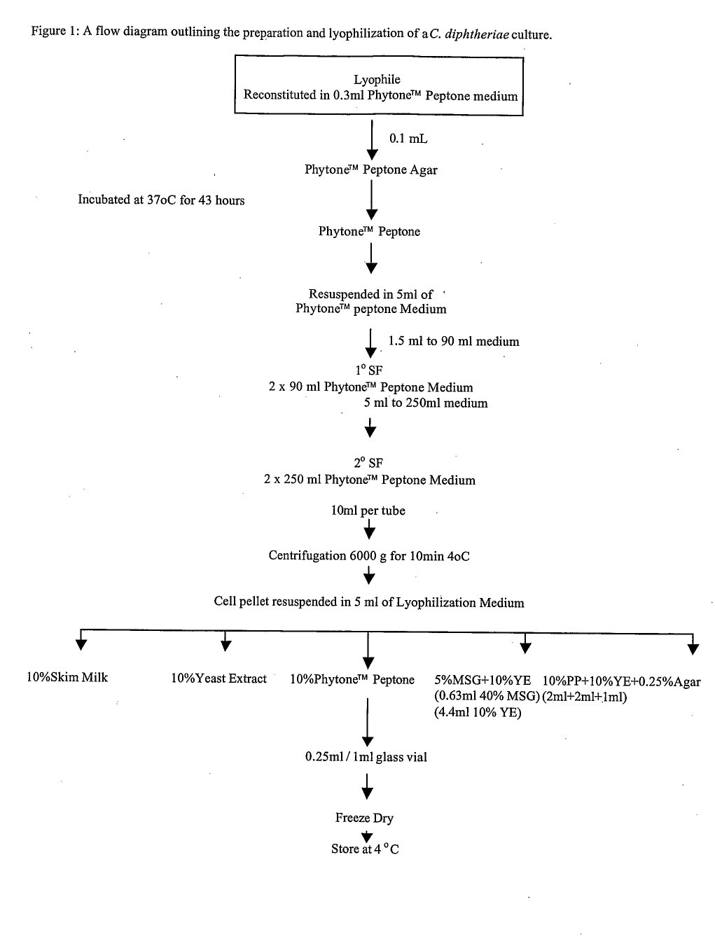

A flow diagram outlining the preparation and lyophilization of C. diphtheriae culture is shown in FIG. 1. A lyophile of C. diphtheriae strain 1M1514N3S was inoculated onto an agar plate containing Phytone™ peptone agar and incubated at 36° C. for 43-48 hours. The composition of Phytone™ peptone medium is described in Tables 1-2 below.

TABLE 1
Composition of the Phytone ™ peptone
medium containing 15 g/L of Phytone ™

| Ingredient | Quantity per Liter | |
|---|---|---|
| Phytone ™ Peptone | 15 | g |
| Acetic acid | 7.2 | mL |
| Maltose | 25 | g |
| Growth Factors | 8 | mL |
| 10% L-Cystine | 2 | Ml |
| 60% Sodium Lactate | 1.7 | Ml |
| PH | 7.5 | |

TABLE 2
Composition of the growth factor solution

| Ingredient | Quantity | |
|---|---|---|
| Magnesium sulphate | 225 | g |
| Beta Alanine | 2.30 | g |
| Pimelic acid | 0.15 | g |
| Zinc sulphate | 0.80 | g |
| Copper sulphate | 0.50 | g |
| Manganese chloride | 0.24 | g |
| Nicotinic acid | 4.6 | g |
| Hydrochloric acid, concentrated | 30 | mL |
| Water for Injection | 1000 | mL |

TABLE 3
A typical analysis of Phytone ™ Peptone as provided
by the manufacturer Difco Laboratories is provided below:
Nitrogen Content/Physical Characteristics

| | |
|---|---|
| Total Nitrogen (TN) (%) | 9.0 |
| Amino Nitrogen (AN) (%) | 2.4 |
| AN/TN | 0.27 |
| Ash (%) | 12.4 |
| Loss on Drying (%) | 1.5 |
| NaCl (%) | 4.0 |
| pH (2% solution) | 7.1 |

Elemental Analysis

| | |
|---|---|
| Calcium (μg/g) | 1001 |
| Magnesium (μg/g) | 2435 |
| Potassium (μg/g) | 31547 |
| Sodium (μg/g) | 34037 |
| Chloride (%) | 0.76 |
| Sulfate (%) | 0.67 |
| Phosphate (%) | 0.64 |

Amino Acid Analysis

| | Free | Total |
|---|---|---|
| Alanine (%) | 0.3 | 2.6 |
| Aspartic Acid (%) | 0.3 | 3.9 |
| Glutamic Acid (%) | 0.3 | 5.9 |
| Histidine (%) | 0.2 | 0.8 |
| Leucine (%) | 0.8 | 2.3 |
| Methionine (%) | 0.2 | 0.2 |
| Proline (%) | 0.1 | 1.8 |
| Threonine (%) | 0.1 | 0.5 |
| Tyrosine (%) | 0.2 | 0.8 |
| Arginine (%) | 0.6 | 2.1 |
| Cystine (%) | 0.4 | Destroyed by hydrolysis |
| Glycine (%) | 0.2 | 1.5 |
| Isoleucine (%) | 0.2 | 1.3 |
| Lysine (%) | 1.2 | 2.4 |
| Phenylalanine (%) | 0.2 | 1.4 |
| Serine (%) | 0.4 | 0.5 |
| Tryptophan (%) | Below level of detection | Destroyed by hydrolysis |
| Valine (%) | 0.1 | 1.5 |

The culture was resuspended in 5 mL of Phytone™ peptone medium and 1.5 ML of the culture transferred to a primary shake flask containing 90 mL of Phytone™ peptone medium containing 0.9 mL of a 1:10 diluted phosphate solution (32% (w/v)) and 0.45 mL of 1:2 diluted calcium chloride solution (53% (w/v)). The culture was incubated at 36° C., 200 rpm for 24 hours. Five mL of the culture was transferred to a secondary shake flask culture containing 250 mL of Phytone™ peptone medium containing 2.5 mL of a 1:10 diluted phosphate solution (32% (w/v)) and 1.25 mL of a 1:2 calcium chloride solution (53% (w/v)). The culture was incubated at 36° C. for a further 24-28 hours. Ten mL of the above secondary shake flask culture was dispensed into five 50 mL sterile screw capped centrifuge tubes and centrifuged at 6 000 ×g for 10 minutes at 4° C.

The supernatant was decanted and the pellet of each tube, re-suspended in 5 mL of one of the following lyophilization media:

a) 10% (w/v) skim milk (Animal Control)

b) 10% (w/v) yeast extract

c) 10% (w/v) Phytone™ peptone

d) 5% (w/v) monosodium glutamate+10% (w/v) yeast extract

e) 10% (w/v) Phytone™ peptone+10% (w/v) yeast extract+0.25% (w/v) agar

The cultures in the above lyophilization medium were dispensed in 0.25 mL amounts in 1 mL glass vials and freeze dried as follows.

Freeze-Drying Cycle

The product temperature was allowed to reach −30° C. and held at that temperature for about 10-12 hours under a vacuum of 120 mT. After 10-12 hours, the product temperature was increased and maintained at 20° C. under a vacuum of 120 mT. The vials are sealed under vacuum and stored at 4° C. The freeze dried cultures were analyzed for viability by measuring colony forming units (CFU/mL) on Columbia blood agar plates.

The results of CFUs obtained before and after freeze-drying for C. diphtheriae strain are shown tabulated in Tables 4, 5, 6 and 7.

TABLE 4
Comparison of CFU counts of the freeze dried cultures
of C. diphtheriae in skim milk and animal component-free
lyophilization medium. The CFU count before freeze-drying
of C. diphtheriae was 6.0 × 10⁹ CFU/mL

| Lyophilization medium | CFU/ml | % Viability |
|---|---|---|
| Skim Milk (Animal Component | 1.24 × 10⁹ | 21 |
| Control) | | |
| MSG + Yeast extract | 1.08 × 10⁹ | 18 |

TABLE 5
Comparison of CFU counts of the freeze dried cultures of
C. diphtheriae in skim milk and animal component-free
lyophilization medium as a function of time. (C. diphtheriae strain)

| Lyophilization | | | | | | |
|---|---|---|---|---|---|---|
| medium | Day 0 | Day 7 | Day 16 | Day 45 | Day 86 | Day 120 |
| Skim Milk | 1.24 × 10⁹ | 5.6 × 10⁷ | 7 × 10⁶ | 3 × 10⁶ | 3 × 10⁶ | 3 × 10⁸ |
| MSG + Yeast | 1.08 × 10⁹ | 9.5 × 10⁸ | 3.2 × 10⁸ | 7.9 × 10⁸ | 3.5 × 10⁸ | 3.4 × 10⁸ |
| Extract | | | | | | |

TABLE 6
Screening of the animal component-free lyophilization medium
and their respective CFU counts in comparison to animal component
lyophilization medium after freeze-drying.

| Freezing Mixture | CFU/ml |
|---|---|
| Skim Milk (animal component) | 1.2 × 10⁷ |
| 10% Yeast extract | 1.9 × 10⁷ |
| 10% Phytone ™ peptone | 1.36 × 10⁸ |
| MSG + Yeast extract | 6.0 × 10⁸ |
| Yeast extract + Phytone ™ | 1.76 × 10⁸ |
| peptone + Agar | |

TABLE 7
Comparison of CFU counts of the freeze dried cultures
of C. diphtheriae in animal component and animal
component-free lyophilization medium.

CFU/mL

| Time (Days) | 10% Skim Milk | 5% MSG + 10% YE |
|---|---|---|
| 0 | 2.04 × 10⁹ | 1.0 × 10⁹ |
| 1 | 2.0 × 10⁷ | 1.22 × 10⁹ |
| 16 | 5 × 10⁶ | 2.96 × 10⁸ |
| 45 | 2.0 × 10⁶ | 1.02 × 10⁹ |
| 86 | 2.0 × 10⁶ | 3.2 × 10⁸ |

The most stable mixture for freeze-drying is the mixture of Yeast extract (10% w/v) with mono sodium glutamate (5% w/v), as shown in Tables 4-7.

### SUMMARY OF THE DISCLOSURE

In summary of this disclosure, there is provide, a lyophilization medium for a microorganism wherein the medium is substantially free of animal-derived products and comprises yeast extract and monosodium glutamate and uses thereof. Modifications are possible within the scope of the invention.

We claim:

1. A lyophilization medium for a microorganism wherein the medium is substantiality free of animal-derived products and comprises yeast extract and monosodium glutamate.

2. The lyophilization medium of claim 1, comprsing about 1-10% (w/v) monosodium glutamate and about 1-10% (w/v) yeast extract.

3. The lyophilization medium of claim 2, comprsing about 5% (w/v) monosodium glutamate and about 10% (w/v) yeast extract.

4. The lyophilization medium of claim 1 or 2 or 3 wherein the microorganism is a strain of bacteria.

5. The lyophilization medium of claim 4 wherein the strain of bacteria is Corynebacterium diphtheriae

6. A method for preparing a freeze-dried culture of a microorganism comprising the steps of:
providing a quantity of the microorganism;
mixing said quanity with a lyophilization medium wherein the medium is substantiality free of animal-derived products and comprises yeast extract and monosodium glutamate to provide a mixture; and
freeze-drying said mixture.

7. The method of claim 4, wherein the lyophilization medium of comprses about 5% (w/v) monosodium glutamate and about 10% (w/v) yeast extract.

8. The method of claim 5, wherein the lyophilization medium of comprses about 1-10% (w/v) monosodium glutamate and about 1-10% (w/v) yeast extract.

9. The method of claim 6 or 7 or 8 wherein freeze-drying of said mixture comprises steps of:
(a) achieving a first temperature of about −30° C. for said mixture to provide a cooled mixture;
(b) maintaining said cooled mixture in a vacuum for a time until said cooled mixture is substantially dry to provide a dried mixture.

10. The method of claim 7 wherein the vacuum is about 120 mT.

11. The method of claim 8 wherein the time is between about 10 and about 12 hours.

12. The method of claim 7 wherein the step of maintaining said cooled mixture in a vacuum for a time until said cooled mixture is substantially dry to provide a dried mixture comprises:
(a) maintaining said cooled mixture in a vacuum for a time of between about 10 and about 12 hours; and
(b) increasing said temperature of about −30° C. to a second temperature of about +20° C.

13. The method of claim 10 wherein the vacuum is about 120 mT.

14. The method of claim 6 or 7 or 8 wherein the microorganism is a strain of bacteria.

15. The method of claim 14 wherein the strain of bacteria is a strain of Corynebacterium diphtheriae

16. A free-dried lyophile comprising cells of a microorganism and a lyophilization medium wherein the medium is substantiality free of animal-derived products and comprises yeast extract and monosodium glutamate.

17. The freeze-dried lyophile of claim 12, wherein the medium comprises about 1-10% (w/v) monosodium glutamate and about 1-10% (w/v) yeast extract.

18. The freeze-dried lyophile of claim 13, wherein the medium comprises about 5% (w/v) monosodium glutamate and about 10% (w/v) yeast extract.

19. The freeze-dried lyophile of claim 16 or 17 or 18 wherein the microorganism is a strain of bacteria.

20. The freeze-dried lyophile of claim 19 wherein the strain of bacteria is a strain of Corynebacterium diphtheriae.
